(19) United States
(12) Patent Application Publication
(10) Pub. No.: US 2002/0176874 A1
(43) Pub. Date: Nov. 28, 2002

(54) COSMETIC COMPOSITIONS WITH HIGH VISCOSITY FOR FEMALE BREAST CARE CONTAINING RIGELIA EXTRACT

(76) Inventors: CHRISTIAN DAMPEIROU, ALLONNE-BONGENOULT (FR); FREDERIC SERIN, PARIS (FR)

Correspondence Address: BIERMAN MUSERLIAN AND LUCAS, 600 THIRD AVENUE, NEW YORK, NY, 10016

(21) Appl. No.: 09/469,530

(22) Filed: Dec. 22, 1999

(30) Foreign Application Priority Data
Jul. 10, 1998 (FR) 98.08917
Jul. 9, 1999 (FR) PCT/FR99/01695

(51) Int. Cl. A61K 7/00

(52) U.S. Cl. 424/401

(57) ABSTRACT

The present invention relates to the field of cosmetics and more especially to compositions meant for care of the female bust. It has as its object new cosmetic compositions with high viscosity meant to be applied to the skin directly or indirectly by means of an adhesive item of fabric attached to an item of ladies' clothing, characterised in that they include one or several extracts of plants with an estrogen type action and an extract of the plant Kigelia africana or Kigelia pinnata in a viscous excipient that is non allergizing and harmless on the toxicological level.

Used for bodily beauty care.

The present invention relates to cosmetics and more particularly to compounds intended for female bust care.

More specifically, its object is new compounds with high viscosity meant to be applied to the skin directly or indirectly by means of a piece of adhesive cloth, attached to an item of ladies' wear.

It concerns in particular new cosmetic compounds for impregnating or coating textile articles, notably items of lingerie or hosiery, especially natural or synthetic fabrics, woven or unwoven, and containing as active element, hormonal preparations of vegetable origin.

The invention relates to cosmetic compounds with an aesthetic effect, characterised by including one or more plant extracts with an estrogen-like action and an extract from the plant Kigelia africana (Bignoniaceous) or Kigelia pinnata in an adhesive support that is pharmacologically inert, the whole being of a fluid viscous consistency placed in store inside an adhesive porous dressing like those of the type transdermic devices.

In fact, it has been found that by placing an adhesive porous dressing on an item of ladies' clothing and in particular on an under-garment such as a brassiere, it was possible to ensure a regular and long-term release of the active ingredients contained in the plant extracts having an estrogen-like action. This effect is strengthened by the addition of an extract from Kigelia africana.

The literature indicates that a certain number of plants such as clover, hop, common ladies' mantle, sage and/or liquorice possess properties of the estrogen type, i.e. they show certain peripheral properties of estrogen hormones without however producing the central effects of hormones as for example the effects on secretion of pituitary hormones (FSH, LH) or on secretion of the Releasing Factors which control pituitary secretion.

These properties are often correlated with the presence in these plants of important contents in phytosterols like sitosterol, stigmasterol, or in saponins likely to convert on contact cutaneous enzymes into androstadien dione, itself a precursor of estrone.

Moreover, the active elements of hop, humulenes and humulone, have slightly estrogenic properties.

The compounds according to the invention can moreover be strengthened by an extract of soya rich in flavons or isoflavons whose effect is added to that of vegetable extracts.

The preparation according to the invention is meant for coating or impregnating or filling the store of the adhesive dressing. According to one modality of the invention, the fluid preparation impregnates the piece of cotton placed at the centre of the adhesive dressing.

According to another form of embodiment, the composition according to the invention impregnates or coats a band of aerated, elasticated cotton tissue protected on the outside by a layer of synthetic or natural cloth, sleek, siliconated, such as unwoven fabric, semi-permeable unwoven polyurethane, or else again a woven band, elastic in length, covered with fine particles of latex.

According to another mode of embodiment of the invention, the support could be a cotton tissue of viscose and elastane covered with a self-adhesive emulsion of the active elements containing moreover latex.

According to another form of embodiment of the invention, the support is formed by a gel of lattice-work polyurethane having about 1 cm thickness on a foam base, the whole being wrapped in a stocking-stitch casing of an allergic polyurethane permeable to the air.

According to another mode of embodiment of the invention, the support is formed by a film of polyurethane, an adhesive expanse with an acrylic polymer base, and a veil of polypropylene placed on the outside of a siliconated protective item.

The support is meant to be attached to a piece of ladies' underwear in contact with skin, such as the brassiere. The composition according to the invention has an aesthetic effect notably by stimulating the development of the female bust. One may also reflect that the presence of flavonosides in the vegetable extracts like luteolin and quercitrin, or their glucosides, has the effect of stimulating the enzymatic activity of tissues as for example that which favours the formation of cross-links between collagen fibres, strengthening their resistance and their firmness. Thus, there is obtained a preparation showing both a positive action on the development of the bust and a positive action on the firmness of tissue.

In the preparations according to the invention, there is incorporated 1 to 10% extract of clover, from 0.5 to 15% of flavosterone (soya extract) rich in flavones, from 0.5 to 10% of extract of hop, from 0.5 to 8% of extract of common ladies' mantle, from 0.25 to 10% of extract of sage, and from 0.15 to 10% extract of liqorice. The extract of Kigelia—and notably the extract in water and butylene glycol (53-47%) is present in quantities ranging from 5 to 25%. Preparations contain also an adhesive agent like acrylic copolymer, marketed under the brand name Ultrez 10, or a copoloymer PEG/PPG (17/6), marketed under the name UCON 75 H450, a thickening agent like chitin in quantities ranging from 10 to 30%, surfactant agents like Tweens or Spans, for example Tween 20, solvents like ethoxydiglycol marketed under the brand Transcutol, binding agents like hydrolysed wheat gluten, marketed under the label Vegetable Tensor LIB, as well as additives like a perfume, antiseptics, preserving agents, antioxydant agents or stabilising agents.

Preparations according to the invention appear in the form of a pasty preparation meant to coat or impregnate a watertight sub-layer attaching to an item of ladies' lingerie. The presence of a thickening agent or adhesive agent helps to confer on this preparation a high viscosity which lets it spread over the sub-layer, either by coating or hot-pouring or by pressing. The adhesive strips thus formed can be cut to the required sizes to be placed on the inner lining of the brassiere.

The preparation dose included in the adhesive strip is calculated so as to provide an efficient duration of contact of several days. The adhesive strip can be easily detached and thus the item of lingerie can be washed as often as one wishes.

The invention relates also to preparations of high viscosity, presented in the form of local compositions meant to be applied to the skin with a basis of plant extracts of estrogenic like action and of extract of the plant Kigelia africana or pinnata, in association with or in ad-mixture with liquid or fluid excipients appropriate for local application.

The appropriate forms are ointments, creams or gels, of a more or less high viscosity according to the place or mode of application. The addition of thickening agents like chitin or acrylic copolymers or copolymers PEG/PPG in variable proportions allows the regulation of the viscosity and rheology of the preparation. According to the varying skin, nature of the epidermis or the measurements of the person concerned, it may be advantageous to use a cream or a gel of greater or lesser viscosity. The addition of ethoxydiglycol, which makes a very good solvent, helps moreover to make the preparation more fluid. Finally, local preparations can be scented with the aid of a vegetable or synthetic perfume that is harmless toxicologically and shows no physical or chemical incompatibilities with the other active elements making up local preparations.

Phytohormonal extracts in local preparations are present at a dosage ranging from 3 to 85%, the thickening agent such as chitin being present at a dosage spanning 10 to 40% in weight. The copolymer product PEG/PPG is used at a concentration spanning 0.75 to 5%. The concentration of a solvent like Transcutol spans 1 to 2.5%. Also, there may be added topical compositions of a dispersing agent such as a non-ionic surfactant, for example a Tween or a Pluronic at a concentration spanning between 0.5 and 1.5%.

Topical preparations can also be added to with an extract of Kigelia africana, preferably the extract in water and butylene glycol (50-50%), at concentrations spanning between 5 to 20%. These compositions are moreover added to by hydrolysed wheat gluten at a concentration of 5 to 20%. The content in water is of the order of 40 to 60% of the total volume.

Topical compositions are meant to be applied to the skin and notably the bust once to four times per day to produce the desired aesthetic effect.

The following examples illustrate the invention without however limiting it.

### EXAMPLE I

Bust gel

| | |
|---|---|
| Acrylic copolymers marketed under the brand name Ultrez 10 | 0, 90 |
| Extract of Kigelia africana in water/butylene glycol | 8 |
| Extract of clover | 3 |
| Flavosterone SB (extract of soya) | 3 |
| Hydrolysed wheat gluten | 9 |
| Polyethylene glycol/polypropylene glycol copolymer marketed | 0.90 |
| under the name UCON 75H450 | |
| Tween 20 (polysorbate 20) | 1 |
| Transcutol | 1, 50 |
| Diazolidinylurea marketed under the name Germaben II | 0, 80 |
| Perfume of Passion fruit | 0, 70 |
| Water ad | 100 g |

### EXAMPLE II

Bust gel

| | |
|---|---|
| Extract of Kigelia africana in water/butylene glycol | 10 |
| Extract of clover | 3 |
| Flavosterone SB | 4 |
| Hydrolysed wheat gluten | 15 |
| Copolymer PEG/PPG 17/6 | 0, 90 |
| Chitin | 15 |
| Acrylic copolymer marketed under the name Ultrez 10 | 0, 90 |
| Transcutol | 2, 50 |
| Tween 20 | 1 |
| Germaben II | 0, 8 |
| Perfume of Passion fruit | 0, 7 |
| Water ad | 100 g |

Extract of Kigelia africana is a plant extract in a mixture of butylene glycol (47%) and water (53%) with a preserving agent added (Phenonip 2,5%). It is a brown-red coloured liquid with a slight aroma. Its density at 20° C. is 1.030±0.020 and its refraction index is 1.395±0.020. Its pH is 5.0 to 6.0. It is miscible in water and alcohol at 60%.

Kigelia africana extract includes steroid flavonoids and saponins. 1 Kg of extract corresponds to 1 Kg of fresh plant.

1. New cosmetic compositions with high viscosity, meant to be applied to the skin directly or indirectly by means of a piece of adhesive fabric attached to an item of ladies' clothing, characterised in that they include one or several plant extracts of plants with an estrogen type action and an extract of the plant Kigelia africana or pinnata, in a viscous excipient that is non allergizing and harmless on the toxicological level.

2. New cosmetic compositions according to claim 1, characterised in that they are placed in store in an adhesive porous dressing like those of the transdermic device type.

3. New cosmetic compositions according to claim 1 or claim 2, in which the plants with estrogen type action are those of a plant chosen from among Clover, Hop, Common Ladies' mantle, Sage or Liquorice.

4. Cosmetic compositions according to one of claims 1 to 3, characterised in that they contain in addition an extract of soya rich in flavones.

5. Cosmetic compositions according to one of the claims 1 to 4, characterised in that they contain the extract of Kigelia in the form of a solution in water and butylene glycol.

6. Cosmetic compositions according to any one of the claims 1 to 5, characterised in that they contain in addition an adhesive agent.

7. Cosmetic compositions according to any one of claims 1 to 6, characterised in that they contain in addition a thickening agent.

8. Cosmetic compositions according to any one of claims 1 to 7, characterised in that they contain in addition surfactants.

9. Cosmetic compositions according to any one of claims 1 to 8, characterised in that they include in addition a solvent.

11. Cosmetic compositions according to any one of claims 1 to 9, characterised in that they include a binding agent.

12. Cosmetic compositions according to any one of claims 1 to 10, characterised in that they include in addition additive substances chosen amongst perfumes, antiseptics, preserving agents, antioxydant agents and stabilising agents.

13. Cosmetic compositions according to any one of the claims 1 to 11 coming in the form of a pasty preparation meant to coat or impregnate a watertight sub-layer attached to an item of ladies' lingerie.

14. Cosmetic compositions according to any of the claims 1 to 12 coming in the form of topical compositions meant for application to the skin.

15. Cosmetic compositions according to claim 13, presenting in the form of ointments, creams or gels.

16. Cosmetic compositions according to any one of claims 1 to 14 in which the content in phythormonal extracts range from 3 to 85%.

17. Cosmetic compositions according to any one of claims 1 to 14 in which the content in Kigelia extract ranges from 5 to 25%.

18. Cosmetic compositions according to any one of claims 1 to 14 in which the content in thickening agent ranges from 0.75 to 40% by weight.

19. Cosmetic compositions according to any one of the claims 1 to 14 in which the content of solvent ranges from 1 to 2.5%.

20. Cosmetic compositions according to any one of claims 1 to 14 in which the content of flavosterone ranges from 0.5 to 15%.
